(19) United States
(12) Patent Application Publication
(10) Pub. No.: US 2025/0050020 A1
(43) Pub. Date: Feb. 13, 2025

(54) A WEARABLE TRANSDERMAL DRUG APPLICATOR

(71) Applicant: COSMOAESTHETICS PTY LTD, Kew (AU)

(72) Inventor: Masoud JAFARZADEH, Kew (AU)

(21) Appl. No.: 18/720,208

(22) PCT Filed: Dec. 14, 2022
(86) PCT No.: PCT/AU2022/051498

§ 371 Date: Jun. 14, 2024

(30) Foreign Application Priority Data
Dec. 17, 2021 (AU) 2021904106

(51) Int. Cl. A61M 5/172

(52) U.S. Cl. CPC A61M 5/1723; A61M 2205/36

(57) ABSTRACT

A wearable transdermal drug applicator has a dock for a liquid drug capsule, a dermal contact face, a delivery channel configured for delivering liquid from the drug capsule through a delivery port of the dermal contact face, a controller and a permeation enhancing subsystem interfacing the dermal contact face. The permeation enhancing subsystem has permeation enhancing elements operatively surrounding the delivery port and which are operative to enhance transdermal drug delivery at the dermal contact face. The elements have a sonicator configured to apply ultrasound to skin contacting the dermal contact face, electrodes configured to configured to apply electric current to the skin contacting the dermal contact face, a heater configured to heat the skin contacting the dermal contact face and red light LEDs configured to apply red light to the skin contacting the dermal contact face.

### FIELD OF THE INVENTION

This invention relates generally to a type of wearable transdermal drug applicator for self-administered drug delivery.

### BACKGROUND OF THE INVENTION

Transdermal delivery of oils, serums, peptides and proteins is a promising alternative to conventional parenteral delivery for self-administered drug therapy.

As hypodermic needle delivery of drug molecules to obtain effective systemic circulation is problematic for self-administration, alternative approaches include minimally invasive micro needle arrays or chemical permeation enhancers of accelerants and sorption promoters.

Other approaches involve wearables transdermal drug applicators configured including U.S. Pat. No. 7,780,981 B2 (Dipierro et al.) 24 Aug. 2010 which discloses a wrist wearable comprising a micro pump or pressurised reservoir to dose the skin with liquid drug, the transdermal delivery of which is enhanced with micro-needles, heat, iontophoresis, sonophoresis or chemical permeation enhancers.

The wrist wearable of Dipierro et al. uses a highly permeable skin contacting membrane to deliver the liquid drug from a reservoir to the skin surface.

WO 1986/0007269 A1 (Drug Delivery Systems Inc) 18 Dec. 1986 and U.S. Pat. No. 5,603,693 A (Frenkel et al.) 18 Feb. 1997 discloses similar transdermal drug applicators which employ an electric circuit created between the applicator and the skin to enhance transdermal drug delivery by electrophoresis or iontophoresis.

The present invention seeks to provide a way to overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a wearable transdermal drug applicator comprising a dock for a liquid drug capsule. The applicator defines a dermal contact face and has a delivery channel configured for delivering liquid from the drug capsule through a delivery port of the dermal contact face.

The applicator further comprises a controller and a permeation enhancing subsystem interfacing the dermal contact face which is controlled by the controller.

The permeation enhancing subsystem comprising permeation enhancing elements operative to enhance transdermal drug delivery at the dermal contact face.

The elements comprising a sonicator configured to apply ultrasound to skin contacting the dermal contact face for sonophoretic effect to enhance transdermal delivery of the liquid. The sonicator may vibrate at a frequency between 1.5-4 kHz.

The elements further comprise electrodes configured to apply mild electric current to the skin contacting the dermal contact face for iontophoretic effect to enhance transdermal delivery of the liquid.

The elements further comprise a heater configured to heat the skin contacting the dermal contact face for vasodilatory and micro-circulation enhancing effect to enhance transdermal drug delivery at the dermal contact face. The heater may comprise an electrical resistive heater element.

The elements further comprise red light LEDs configured to apply red light to the skin contacting the dermal contact face for vasodilatory and micro-circulation enhancing effect to further enhance transdermal drug delivery at the dermal contact face.

The red light LEDs may be configured to emit a wavelength centred at approximately 670 nm optimised for vasodilatory and micro-circulation enhancing effect and which may penetrate through the epidermis to the underlying dermis.

The present particular configuration comprising the permeation enhancing elements operatively surrounding the delivery port through the dermal contact face allows unobstructed operative effect of each of the permeation enhancing elements and further allows the controller to individually or synchronously control the permeation enhancing elements to control the rate of drug delivery.

Specifically, the delivery of the liquid drug via the delivery port through the dermal contact face avoids using permeable skin contacting membranes as are taught by Dipierro et al. thereby allowing unobstructed light propagation of the red light LEDs and without heat conductivity attenuation of the heating elements or sound attenuation of the sonicator.

The controller may be programmed with a delivery program and wherein the controller is configured for controlling the sequence of operation of the elements according to the delivery program.

The delivery program may comprise a heat sequence during which heater is controlled accordingly, a current sequence during which the electrodes are controlled, an ultrasound sequence during which the sonicator is controlled and a light sequence during which the red light LEDs are controlled.

The delivery program may include intermittent delivery wherein the controller is configured for controlling the operation and nonoperational at least one of the elements accordingly. For example, the controller may be configured for controlling simultaneous operation and nonoperational of the sonicator and red light LEDs to control or maintain the vasodilatory and/or micro-circulatory effect to thereby control the transdermal rate of delivery accordingly.

In embodiments, the controller may read delivery program instructions directly from the capsule specific to the type of drug being delivered.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a dermal contact face of a wearable transdermal drug applicator in accordance with an embodiment;

FIG. 2 shows a side cross-sectional view of the applicator;

FIG. 3 shows a perspective view of applicator in accordance with an embodiment;

FIG. 4 shows a rear perspective view of the applicator in accordance with a further embodiment;

FIGS. 5 and 6 show perspective views of the applicator with lid open and closed respectively;

FIG. 7 shows exemplary circuitry of the applicator;

FIG. 8 shows an exemplary timing sequence of a delivery program in accordance with an embodiment; and

FIG. 9 shows an exemplary flow diagram of the delivery program.

### DESCRIPTION OF EMBODIMENTS

A wearable transdermal drug applicator 100 comprises a small form factor housing 101 held by wrist straps 102.

The applicator 100 comprises a dock 103 for drug capsule 104. The drug capsule 104 contains a drug liquid 105 therein.

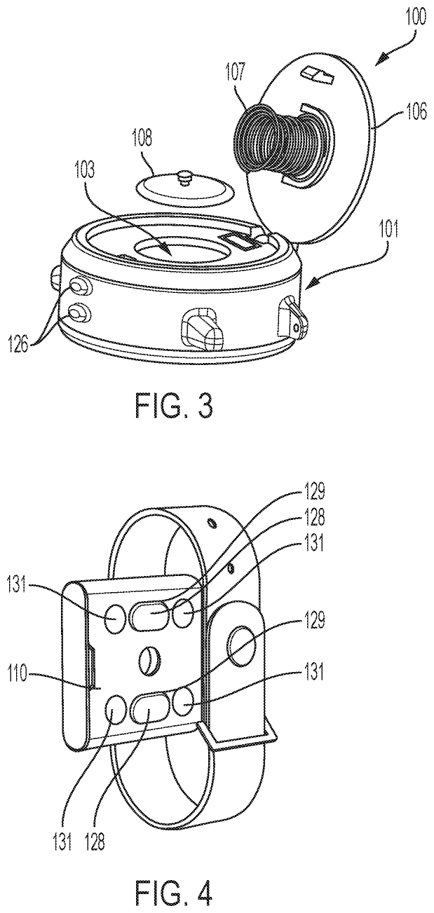

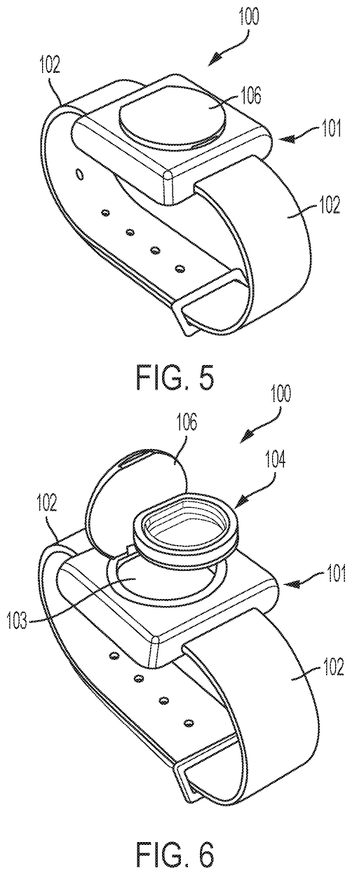

According to the embodiment shown in FIGS. 3, 5 and 6, the applicator 100 has a lid 106 covering the dock 103.

As is shown in FIG. 3, the lid 106 may comprise a compression spring 107 which may push a press plate 108 onto the drug capsule 104 to pressurise the liquid 105 therein.

The drug capsule 104 may comprise a squeezable plastic body able to be squeezed under pressure of the push plate 108. Sidewalls of the capsule 104 may be compressible or deformable to allow vertical compression by the press plate 108 to thereby pressurise the liquid 105.

The applicator 100 has a dermal contact face 110 which contacts skin 113 of the wrist when the applicator 100 is worn. Preferably the applicator 100 is worn so that the dermal contact face 110 contacts the more sensitive skin under the wrist.

The applicator 100 defines a delivery channel 111 configured for delivering the liquid 105 from the drug capsule 104 through a delivery port 112 of the dermal contact face 110.

The dock 103 may comprise puncture formations 109 which may puncture through a bottom wall of the drug capsule 104 to allow the liquid 105 to escape therefrom via the delivery port 112 and across the skin 113 as is shown in FIG. 2. Whereas one delivery port 112 is shown in the illustrative embodiments, in alternative embodiments, a plurality of delivery port 112 may be provided through the dermal contact face 110.

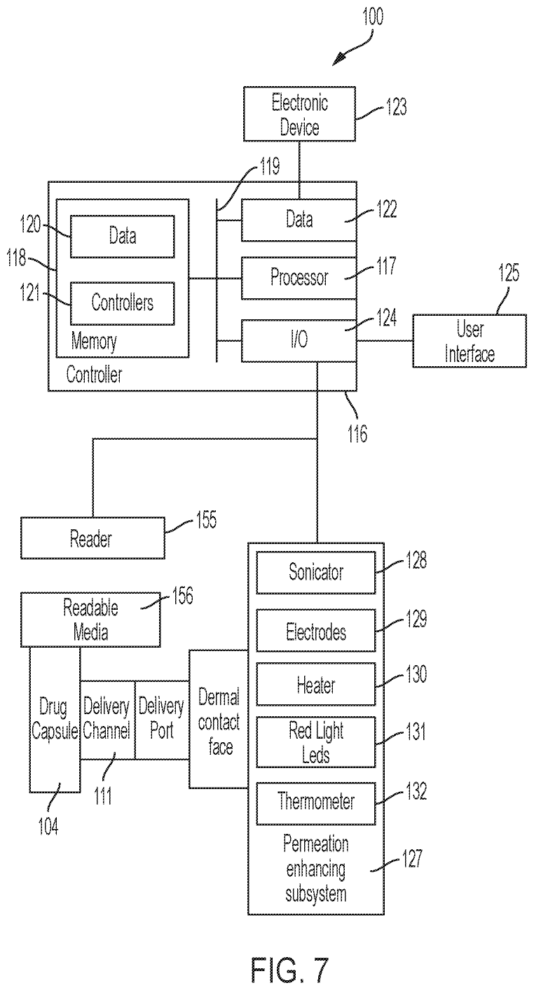

According to FIG. 7, the applicator 100 comprises a controller 116. At a 100 may comprise a rechargeable battery (not shown) to power the controller 116 and other electrical componentry of the applicator 100.

The controller 116 comprises a processor 117 for processing digital data. A memory device in operable communication with the processor 117 via system bus 119 is configured for storing digital data 120 and computer program code instructions. In use, the processor 117 fetches these computer program code instructions and associated data 120 from the memory 118 for interpretation and execution of the functionality described herein, including implementation of delivery programs. The computer program code instructions may be logically divided into a plurality of computer program code instruction controllers.

The controller 116 may take the form of a low-power firmware-based microcontroller. The controller 116 may comprise a data interface 122 for sending and receiving data across a data network, such as a short-range wireless data network such as a Bluetooth network. In this way, the controller 116 may communicate with an electronic device 123.

The electronic device 123 may take the form of a mobile communication device having a software application installed and executed by the electronic device 123 to interface with the controller 116, such as for the display of information, transmittal of control instructions and the like. The controller 116 may further comprise an I/O interface 124 for interfacing with various peripherals, including a user interface 125.

As shown in FIG. 3, the user interface 125 may comprise a plurality of control buttons 126.

The I/O interface 124 may further interface a permeation enhancing subsystem 127. The permeation enhancing subsystem 127 interfaces the dermal contact face 110 and comprises a plurality of permeation enhancing elements operative to enhance transdermal drug delivery at the dermal contact face 110.

The permeation enhancing elements operatively surround the delivery port 112 to enhance transdermal delivery of liquid 105 escaping therefrom.

These elements comprise a sonicator 128 configured to apply ultrasound 134 to the skin 113 contacting the dermal contact face 110 for sonophoretic effect to enhance transdermal delivery of the liquid 105. The sonicator 128 may vibrate at a frequency between 1.5-4 kHz.

The elements further comprise electrodes 129 configured to apply a mild electric current 133 to the skin 113 for iontophoretic effect to enhance transdermal drug delivery at the dermal contact face 110.

The elements further comprise a heater 130 configured to apply heat 135 to the skin 113 contacting the dermal contact face 110 for vasodilatory and micro-circulation enhancing effect.

The heater 135 may comprise an electrical resistive heater element to enhance transdermal drug delivery at the dermal contact face 110.

The elements further comprise red light LEDs 131 configured to apply red light 136 to the skin 113 contacting the dermal contact face 110.

The red light LEDs 131 may be configured to emit wavelength centred at approximately 670 nm for vasodilatory and micro-circulation enhancing effect and which may penetrate through the epidermis 114 to the dermis 115.

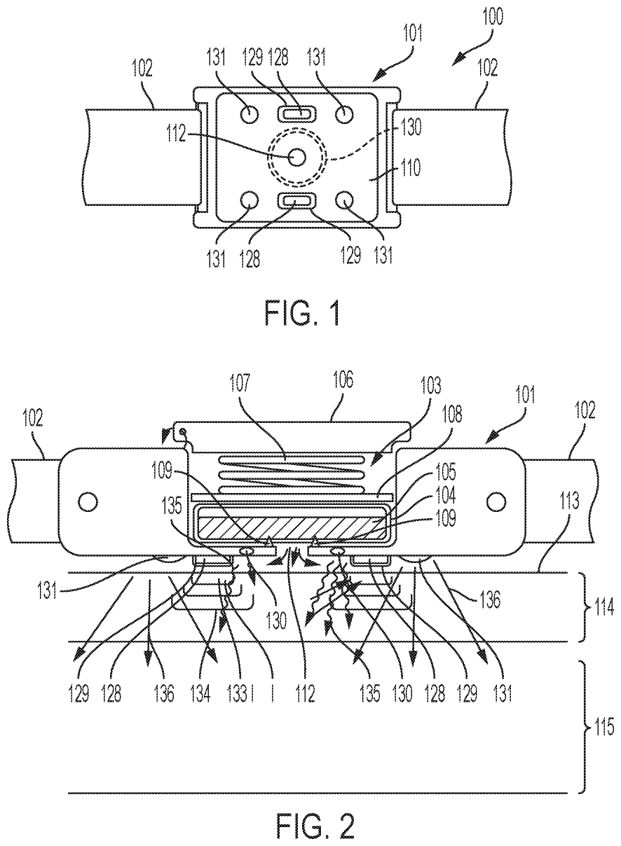

As is shown in FIG. 1, these elements may substantially concentrically surround the delivery port 112 so as to concentrate their respective effect on the liquid 105 escaping via the delivery port 112. In embodiments where in the dermal contact face 110 comprises a plurality of delivery port 112, the positioning of the elements may yet operatively interface the delivery port 112 to enhance transdermal delivery of liquid 105 escaping therefrom.

In the embodiment shown in FIG. 1, the dermal contact face 110 comprises a quadrant of red light LEDs 131.

Furthermore, the heater 130 may substantially concentrically surround the delivery port 112.

Furthermore, as is shown in FIG. 1, the electrodes 129 and sonicators 128 may be conjoined as a unitary assembly comprising a central sonicator 128 and a peripheral metallic electrode 129.

The controller 116 may be programmed with a delivery program and wherein the controller 116 is configured for controlling the sequence of operation of the elements according to the delivery program.

In embodiments, the delivery program encodes an intermittent delivery sequence and wherein the controller 116 is configured for controlling the operation and nonoperation of at least one of the elements accordingly. The controller 116 may be configured for controlling the operation and nonoperation of at least two of the elements simultaneously.

The intermittent delivery sequence may be used by the applicator 100 to maintain or control vasodilatory or microcirculatory effect.

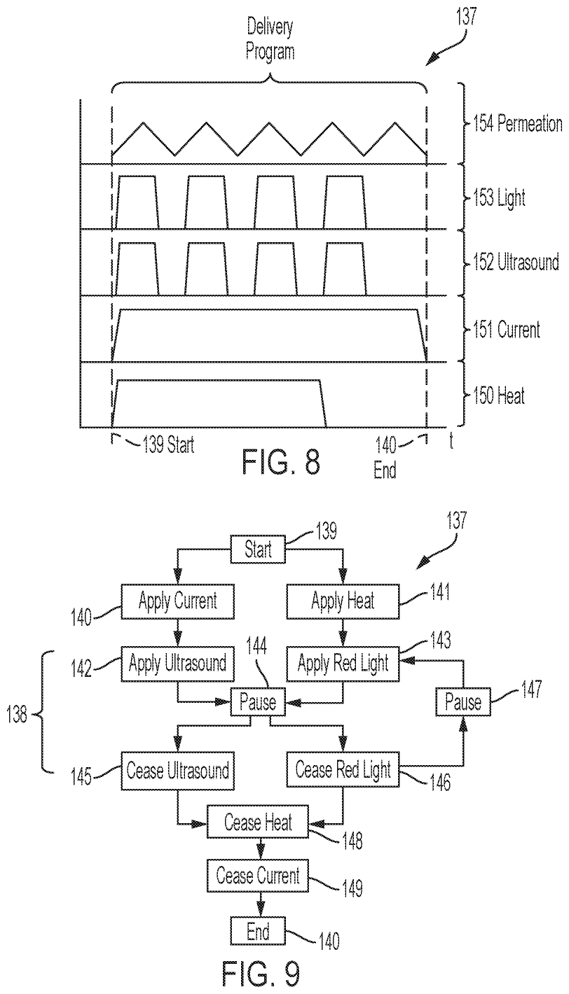

FIGS. 8 and 9 show an exemplary delivery program 137 which includes an intermittent delivery sequence 138.

The program 137 shown in FIG. 8 is according to a timescale and comprises a heat sequence 150 during which heater 130 is controlled accordingly, a current sequence 151 during which the electrodes 129 are controlled, an ultrasound sequence 152 during which the sonicator 128 is controlled and a light sequence 153 during which the red light LEDs 131 are controlled.

At the start 139 of the delivery program, the controller 116 may apply current at step 140 and apply heat at step 141.

The delivery program 137 of FIG. 8 shows the rate of transdermal delivery 154.

Shortly after the start, as the skin 113 is heated, the rate of delivery increases according to the vasodilatory and micro-circulation enhancing heating effect.

The program 137 may include the intermittent delivery sequence 138 to control the transdermal rate of delivery of the liquid drug 105.

The intermittent delivery sequence 138 may be used to maintain or control vasodilatory and microcirculatory effect. Specifically, according to the exemplary intermittent delivery sequence 138 shown in FIG. 9, the controller 116 may execute a sequence wherein ultrasound is applied at step 142 and red light is applied at step 143. After a pause 144 (such as of approximately two minutes for example), the intermittent delivery sequence 138 may comprise ceasing the ultrasound at step 145 and ceasing the red light at step 146. After a further pause 147 (such as a similar two-minute pause), the controller 116 may gain apply ultrasound at step 142 and apply red light at step 143.

Such intermittent delivery sequence 138 may repeat a number of times. As is shown in FIG. 8, the intermittent delivery sequence 138 is shown as repeating four times.

At step 149, the controller 116 may cease application of heat. As is shown in FIG. 7, in embodiments, the I/O interface 116 may interface a thermometer 132 which measures skin temperature at the dermal contact face 110. The controller 116 may control the operation of the thermometer 142 beneath a setpoint, such as approximately 40.5° C. As such, when the skin temperature reaches the setpoint, the controller 116 ceases application of heat at step 148.

At the end of the program 140, the controller 116 may cease application of current at step 149.

An exemplary transdermal delivery program 110 may take approximately 10 minutes.

In embodiments, and as is further shown in FIG. 7, the I/O interface 116 may interface a reader 155 which is configured to read data from readable media 156 of the drug capsule 104. The reader 155 may read the readable media 156 optically such as wherein the readable media 156 comprises a two-dimensional visible encoding thereon which is optically read by the reader 155. In alternative embodiments, the reader 155 may operate using near field communication wherein the readable media 156 comprises a near field communication tag encoding information.

Readable media 156 may encode operational parameters for the delivery program 137, such as the duration and frequency of the operational sequences 150-153. Alternatively, the readable media 156 may encode a type of drug wherein the controller 116 obtain such operational sequences 150-153 from a lookup table within memory 118.

In embodiments, the controller 116 may be configured to detect skin contact by measuring skin electrical conductivity between the electrodes prior operating the permeation enhancing subsystem 127. Furthermore, the controller 116 may be configured to detect delivery of the liquid 105 via the delivery port 112 by measuring electrical conductivity through the liquid between the electrodes 128.

The controller 116 may further configured to detect when the liquid 105 has been depleted according to the electrical conductivity. Detecting that the liquid 105 has been depleted, the controller 116 may output a single accordingly, such as via an audible buzzer, digital display or the like.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A wearable transdermal drug applicator comprising:
a dock for a liquid drug capsule;
a dermal contact face;
a delivery channel configured for delivering liquid from the drug capsule through a delivery port of the dermal contact face;
a controller;
a permeation enhancing subsystem interfacing the dermal contact face, the permeation enhancing subsystem comprising permeation enhancing elements operatively surrounding the delivery port and which are operative to enhance transdermal drug delivery at the dermal contact face, the elements comprising:
a sonicator configured to apply ultrasound to skin contacting the dermal contact face;
electrodes configured to configured to apply electric current to the skin contacting the dermal contact face;
a heater configured to heat the skin contacting the dermal contact face; and
red light LEDs configured to apply red light to the skin contacting the dermal contact face and wherein the controller is programmed with a delivery program and wherein the controller is configured for controlling the operation of the elements according to the delivery program.

2. The applicator as claimed in claim 1, wherein the LEDs are configured to emit a wavelength centred at approximately 670 nm.

3. The applicator as claimed in claim 1, wherein the heater is an electrical resistive heater element.

4. The applicator as claimed in claim 1, wherein the controller is configured for controlling the sequence of operation of the elements according to the delivery program.

5. The applicator as claimed in claim 4, wherein the delivery program comprises a heat sequence during which heater is controlled accordingly, a current sequence during which the electrodes are controlled, an ultrasound sequence during which the sonicator is controlled and a light sequence during which the red light LEDs are controlled.

6. The applicator as claimed in claim 4, wherein the delivery program encodes an intermittent delivery sequence and wherein the controller is configured for controlling operation and non-operation of at least one of the elements accordingly.

7. The applicator as claimed in claim 6, wherein the controller is configured for controlling the operation of at least one of the elements according to the intermittent delivery sequence to control vasodilatory effect thereof.

8. The applicator as claimed in claim 6, wherein the controller is configured for controlling the operation of at least two of the elements according to the intermittent delivery sequence to control vasodilatory effect thereof.

9. The applicator as claimed in claim 8, wherein the controller is configured for controlling simultaneous operation and non-operation of the sonicator and LEDs.

10. The applicator as claimed in claim 1, wherein the controller is configured to cease operation of the heater prior completion of the delivery program.

11. The applicator as claimed in claim 10, wherein the device further comprises a thermometer interfacing the dermal contact face and wherein the controller is configured for controlling the operation of the heater according to a temperature setpoint.

12. The applicator as claimed in claim 11, wherein the controller is configured for operating the heater at a temperature setpoint of less than a maximum temperature setpoint.

13. The applicator as claimed in claim 1, wherein the applicator comprises a reader configured to read delivery program instructions encoded by the drug capsule.

14. The applicator as claimed in claim 13, wherein the delivery program instructions encode a heat sequence during which heater is controlled accordingly, a current sequence during which the electrodes are controlled accordingly, an ultrasound sequence during which the sonicator is controlled accordingly and a light sequence during which the red light LEDs are controlled accordingly.

15. The applicator as claimed in claim 1, wherein the controller is configured to detect skin contact by measuring skin electrical conductivity between the electrodes prior operating the permeation enhancing subsystem.

16. The applicator as claimed in claim 1, wherein the controller is configured to detect delivery of the liquid via the delivery channel by measuring electrical conductivity through the liquid between the electrodes.

17. The applicator as claimed in claim 16, wherein the controller is configured to detect when the liquid has been depleted according to the electrical conductivity.

18. The applicator as claimed in claim 1, wherein the applicator comprises a unitary assembly of a sonicator and an electrode.

19. The applicator as claimed in claim 1, wherein the permeation enhancer elements substantially concentrically surround the delivery port.

20. The applicator as claimed in claim 1, wherein the applicator is a wrist worn device which comprises a wrist strap connected either side thereof without obstructing the elements.

21. The applicator as claimed in claim 1, wherein the dock comprises puncture formations which puncture the capsule when the capsule is inserted into the dock.

22. The applicator as claimed in claim 21, wherein the capsule comprises compressive sidewalls configured to deform when the capsule is pressed to thereby pressurise liquid contents therein.

23. The applicator as claimed in claim 12, wherein the dock comprises a compression spring therein which compresses the drug capsule.
